(19) United States
(12) Patent Application Publication
(10) Pub. No.: US 2017/0003296 A1
(43) Pub. Date: Jan. 5, 2017

(54) METHOD FOR DETECTING HISTONE MODIFICATIONS IN NUCLEOSOMES

(71) Applicants: Mark Edward ECCLESTON, London (GB); SINGAPORE VOLITION PTE LIMITED, Singapore (SG)

(72) Inventor: Mark Edward ECCLESTON, London (GB)

(73) Assignee: SINGAPORE VOLITION PTE LIMITED, Singapore (SG)

(21) Appl. No.: 14/770,796

(22) PCT Filed: Feb. 27, 2014
(86) PCT No.: PCT/EP2014/053852

§ 371 Date: Aug. 26, 2015

Related U.S. Application Data
(60) Provisional application No. 61/770,893, filed on Feb. 28, 2013.

(30) Foreign Application Priority Data
Feb. 28, 2013 (GB) 1303575.3

(51) Int. Cl. G01N 33/68

(52) U.S. Cl. CPC G01N 33/6875; G01N 2800/52

(57) ABSTRACT

The invention relates to a method for detecting and measuring the presence of mono-nucleosomes and oligo-nucleosomes and nucleosomes that contain particular histone modifications and the use of such measurements for the detection and diagnosis of disease. The invention also relates to a method of identifying histone modification biomarkers for the detection and diagnosis of disease and to said histone modification biomarkers identified by said method.

### FIELD OF THE INVENTION

The invention relates to a method for detecting and measuring the presence of mono-nucleosomes and oligo-nucleosomes and nucleosomes that contain particular histone modifications and the use of such measurements for the detection and diagnosis of disease. The invention also relates to a method of identifying histone modification biomarkers for the detection and diagnosis of disease and to said histone modification biomarkers identified by said method.

### BACKGROUND OF THE INVENTION

The human body comprises several hundred cell types. All of these cell types contain the same genome but have widely different phenotypes and different functions in the body. This phenotypic diversity is due to the differential expression of the genome in different cell types. The control of differential gene expression is not entirely understood but the basic mechanisms include gene regulation by a number of interconnected epigenetic signals associated with the gene, including control of the chromatin packing as euchromatin or heterochromatin, control of nucleosome positioning and nuclease accessible sites, methylation of DNA and variation in the structure of the nucleosomes around which the DNA is wrapped.

The nucleosome is the basic unit of chromatin structure and consists of a protein complex of eight highly conserved core histones (comprising of a pair of each of the histones H2A, H2B, H3, and H4). Around this complex are wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, 2007).

The structure of nucleosomes can vary by Post Transcriptional Modification (PTM) of histone proteins and by the inclusion of variant histone proteins. PTM of histone proteins typically occurs on the tails of the core histones and common modifications include acetylation, methylation or ubiquitination of lysine residues as well as methylation of arginine residues and phosphorylation of serine residues and many others. Histone modifications are known to be involved in epigenetic regulation of gene expression (Herranz and Esteller, 2007). The structure of the nucleosome can also vary by the inclusion of alternative histone isoforms or variants which are different gene or splice products and have different amino acid sequences. Histone variants can be classed into a number of families which are subdivided into individual types. The nucleotide sequences of a large number of histone variants are known and publicly available for example in the National Human Genome Research Institute NHGRI Histone DataBase (Marino-Ramirez, L., Levine, K. M., Morales, M., Zhang, S., Moreland, R. T., Baxevanis, A. D., and Landsman, D. The Histone Database: an integrated resource for histones and histone fold-containing proteins. Database Vol. 2011. (Submitted) and http://genome.nhgri.nih.gov/histones/complete.shtml), the GenBank (NIH genetic sequence) DataBase, the EMBL Nucleotide Sequence Database and the DNA Data Bank of Japan (DDBJ).

Normal cell turnover in adult humans involves the creation by cell division of some 10¹¹ cells daily and the death of a similar number, mainly by apoptosis. During the process of apoptosis chromatin is broken down into mononucleosomes and oligonucleosomes which are released from the cells. Under normal conditions the level of circulating nucleosomes found in healthy subjects is reported to be low. Elevated levels are found in subjects with a variety of conditions including many cancers, auto-immune diseases, inflammatory conditions, stroke and myocardial infarction (Holdenreider & Stieber, 2009).

Mononucleosomes and oligonucleosomes can be detected by Enzyme-Linked ImmunoSorbant Assay (ELISA) and several methods have been reported (Salgame et al, 1997; Holdenrieder et al, 2001; van Nieuwenhuijze et al, 2003). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody. Using these assays workers in the field report that the level of nucleosomes in serum is higher (by up to an order of magnitude) than in plasma samples taken from the same patients. This is also true for serum and plasma measurements of DNA made by PCR (Holdenrieder et al, 2005). The reason for this is not known but the authors speculate that it may be due to additional release of DNA during the clotting process. However, we have found that the results of nucleosome ELISA assays of the current art do not agree with each other. Furthermore, although most circulating DNA in serum or plasma is reported to exist as mono-nucleosomes and oligo-nucleosomes (Holdenrieder et al, 2001), measured levels of nucleosomes and DNA in serum or plasma do not agree well. The correlation coefficient between ELISA results for circulating cell free nucleosome levels and circulating DNA levels as measured by real time PCR (Polymerase Chain Reaction) has been reported to be r=0.531 in serum and r=0.350 in plasma (Holdenrieder et al, 2005).

Current nucleosome ELISA methods are used in cell culture, primarily as a method to detect apoptosis (Salgame et al, 1997; Holdenrieder et al, 2001; van Nieuwenhuijze et al, 2003), and are also used for the measurement of circulating cell free nucleosomes in serum and plasma (Holdenrieder et al, 2001). Cell free serum and plasma nucleosome levels released into the circulation by dying cells have been measured by ELISA methods in studies of a number of different cancers to evaluate their use as a potential biomarker (Holdenrieder et al, 2001). Mean circulating nucleosome levels are reported to be high in most, but not all, cancers studied. The highest circulating nucleosome levels were observed in lung cancer subjects. The lowest levels were observed in prostate cancer, which were within the normal range of healthy subjects. However, patients with malignant tumours are reported to have serum nucleosome concentrations that varied considerably and some patients with advanced tumour disease were found to have low circulating nucleosome levels, within the range measured for healthy subjects (Holdenrieder et al, 2001). Because of this and the variety of non-cancer causes of raised nucleosome levels, circulating nucleosome levels are not used clinically as a biomarker of cancer (Holdenrieder and Stieber, 2009). Surprisingly we have shown that many cancer subjects whose circulating nucleosome levels are low or undetectable as measured by these nucleosome ELISA methods of the current art, do in fact have raised levels of circulating cell free nucleosomes. We have designed and demonstrated novel ELISA methods for nucleosomes that detect nucleosomes not detected by ELISA methods of the current art.

Epigenetic control of gene expression in cells is mediated in part by modifications to DNA nucleotides including the cytosine methylation status of DNA. It has been known in the art for some time that DNA may be methylated at the 5 position of cytosine nucleotides to form 5-methylcytosine. Methylated DNA in the form of 5-methylcytosine is reported to occur at positions in the DNA sequence where a cytosine nucleotide occurs next to a guanine nucleotide. These positions are termed “CpG” for shorthand. It is reported that more than 70% of CpG positions are methylated in vertebrates (Pennings et al, 2005). Regions of the genome that contain a high proportion of CpG sites are often termed “CpG islands”, and approximately 60% of human gene promoter sequences are associated with such CpG islands (Rodriguez-Paredes and Esteller, 2011). In active genes these CpG islands are generally hypomethylated. Methylation of gene promoter sequences is associated with stable gene inactivation. DNA methylation also commonly occurs in repetitive elements including Alu repetitive elements and long interspersed nucleotide elements (Herranz and Estellar, 2007; Allen et al, 2004).

The involvement of DNA methylation in cancer was reported as early as 1983 (Feinberg and Vogelstein, 1983). DNA methylation patterns observed in cancer cells differ from those of healthy cells. Repetitive elements, particularly around pericentromeric areas, are reported to be hypomethylated in cancer relative to healthy cells but promoters of specific genes have been reported to be hypermethylated in cancer. The balance of these two effects is reported to result in global DNA hypomethylation in cancer cells (Rodriguez-Paredes; Esteller, 2007).

Hypermethylation of certain specific genes can be used as a diagnostic biomarker for cancers. For example a method reported for detection of hypermethylation of the Septin 9 gene by PCR amplification of DNA extracted from plasma was reported to detect 72% of colon cancers with a false positive rate of 10% (Grutzmann et al, 2008). The DNA methylation status of specific genes or loci is usually detected by selective bisulphite deamination of cytosine, but not 5-methylcytosine, to uracil, leading to a primary DNA sequence change that can be detected by sequencing or other means (Allen et al, 2004).

Global DNA hypomethylation is a hallmark of cancer cells (Estellar 2007 and Hervouet et al, 2010). Global DNA methylation can be studied in cells using immunohistochemistry (IHC) techniques. Alternatively the DNA is extracted from the cells for analysis. A number of methods have been reported for the detection of global methylation in DNA extracted from cells including restriction digestion and nearest-neighbour analysis, fluorescent assays using chloracetaldehyde, inverse determination by methylation of all CpG sites using DNA methyltransferase in conjunction with tritium-labelled S-adenosyl methionine to calculate the amount of unmethylated CpG and digestion of DNA into single nucleotides for analysis by high-performance liquid chromatography, thin-layer chromatography, or liquid chromatography followed by mass spectroscopy. The disadvantages of these methods are that they are labour intensive and/or require large amounts of good quality extracted DNA (Allen et al 2004). PCR based methods involving bisulfite deamination overcome the need for large amounts of DNA but must amplify specific genome regions, typically repetitive sequences, as indicative of the total genome content of 5-methylcytosine (Allen et al 2004). These methods for global DNA methylation measurement have been used to study DNA extracted from a variety of cells and tissues. Some workers have studied DNA extracted from white blood cells in whole blood as this is easier to obtain in a minimally-invasive manner (Moore et al, 2008; Ting Hsiung et al, 2007; Mansour et al, 2010). Liquid Chromatography with mass spectrometry is considered the gold standard for global DNA methylation measurement but it is costly, and the DNA must be digested to the single nucleotide level prior to analysis (Vasser et al, 2009).

Recent methods for the estimation of global DNA methylation include ultra high-pressure liquid chromatography with mass spectrometry of hydrolysed DNA extracted from tissue (Zhang et al, 2011) and a methylation-specific digital sequencing (MSDS) method (Ogoshi et al 2011). A classical competitive immunoassay for global DNA methylation (as well as a similar assay for global 5-hydroxymethylcytosine methylation) has been described. In this method DNA extracted from cells or tissues is added to a microtitre well coated with a 5-methylated cytidine conjugate, an anti-5-methylcytidine antibody is added and the distribution of antibody binding between the coated 5-methylcytidine conjugate and the methylated DNA in the extracted sample is compared to that of known standards to estimate the global DNA methylation level present in the sample (Cell Biolabs, 2011). In another immunoassay like method DNA extracted from tissues or from plasma or serum samples is coated to a microtitre well and methylated DNA is detected using an anti-5-methylcytosine antibody (Vasser, et al, 2009; Epigentek, 2009). A disadvantage of these methods is that they require extraction of DNA involving the denaturation and removal of all nucleosome and chromatin structure from the DNA. They are not suited for example; for the direct measurement of global DNA methylation in biological fluids such as tissue lysate, blood, plasma or serum without a DNA extraction step.

5-hydroxymethyl modification of cytosine bases in DNA has also been reported. The role of 5-hydroxymethylation is not yet well understood but it appears to be involved in gene regulation (Stroud et al, 2011).

Current methods for the detection of global DNA methylation involve extraction or purification of the DNA and are not suitable for rapid, high throughput, low cost, minimally-invasive diagnostic methods. Similarly, analysis of DNA for other modified or unusual bases (for example uracil, inosine, xanthine, and hypoxanthine) can only be investigated by the analysis of substantially pure or extracted DNA. Such analysis cannot be carried out directly in complex biological media such as tissue lysate, blood, plasma or serum.

Histone variants (also known as histone isoforms) are also known to be epigenetic regulators of gene expression (Herranz and Esteller, 2007). Histone variants have been studied in vivo and in vitro using a variety of techniques including knock-down studies of the gene encoding a particular variant (for example using RNAi knock-down), chromatin immunoprecipitation, stable isotope labeling of amino acids and quantitative mass spectrometry proteomics, immunohistochemistry and Western Blotting (Whittle et al, 2008; Boulard et al, 2010; Sporn et al, 2009; Kapoor et al, 2010; Zee et al, 2010; Hua et al, 2008).

Immunohistochemistry studies of histone variant expression in tissue samples removed at surgery or by biopsy from subjects diagnosed with lung cancer, breast cancer and melanoma have been reported. These immunohistochemistry studies report that staining of histone macroH2A (mH2A) and H2AZ variants in resected cancer tissue samples may have prognostic application in these cancers (Sporn et al, 2009, Hua et al, 2008, Kapoor et al, 2010). One disadvantage of immunohistochemical methods for clinical use is that tissue sample collection is invasive involving surgery or biopsy. Another disadvantage of immunohistochemistry methods is that they are unsuited for early diagnosis or for screening diagnostics as a reasonable expectation of the disease must usually already exist before a biopsy or tissue resection is made. Minimally invasive blood ELISA tests are suitable for a wider range of applications and would overcome these disadvantages and be preferable for the patient as well as faster, lower cost and more high-throughput for the healthcare provider.

However, cell free nucleosomes containing particular nucleotides, modified nucleotides or histone variants have not been measured in blood or any other medium and no such measurements have been suggested or contemplated. No studies on the presence or absence of nucleotides, modified nucleotides or histone variants in cell free nucleosomes in blood have been reported nor whether they have value as blood biomarkers of disease. There are currently no methods for the detection or measurement of nucleotides, modified nucleotides or histone variants in intact cell free nucleosomes.

Nucleosome position and nucleosome structure (in terms of both constituent histone protein variant and PTM structures) are also known to mediate epigenetic signaling. Examples of post translation include, but are not limited to, histone modifications including lysine mono-, di- and tri-methylation, lysine acetylation, Arginine mono-methylation and symmetric or asymmetic di-methylation, citrullination, ubiquitinylation, serine or threonine phosphorylation and proline isomerization. It will be recognized by those skilled in the art that these and other histone posttranslational modifications can exert activating or repressive effects on gene expression.

ELISA methods for the detection of histone PTMs are also known in the art. ELISA methods for PTM detection in free histone proteins (not attached to other histones and DNA in a nucleosome complex) are used for the detection of PTMs in histones extracted, usually by acid extraction, from cell lysates. Immunoassay for the detection of PTMs in circulating cell free nucleosomes has been reported (Bawden et al, 2005). In this method cell free nucleosomes in blood, serum or plasma are first immobilized with a first antibody which detects whole nucleosomes and then reacted with anti-PTM antibodies. A method for ELISA detection of histone PTMs in purified nucleosomes directly coated to microtitre wells has recently been reported (Dai et al, 2011). In this method nucleosomes obtained by digestion of chromatin extracts from cultured cells are coated directly to microtitre wells and reacted with anti-PTM antibodies. It will be clear to those skilled in the art that this method requires relatively pure nucleosome samples and is not suitable for the direct measurement of histone PTMs in complex biological media such as blood or serum.

A modified chromatin immunoprecipitation (ChIP) method for the detection of a histone PTM (H3K9Me, histone H3 monomethylated at lysine residue K9) in cell free nucleosomes associated with a particular DNA sequence has been reported in plasma. The level of sequence specific histone methylation was reported to be independent of the concentration of circulating nucleosomes (Deligezer et al, 2008).

It is therefore an object of the invention to provide improved methods for detecting and measuring histone modifications.

### SUMMARY OF THE INVENTION

According to a first aspect of the invention there is provided a method for detecting the presence of a nucleosome containing one or more histone modifications in a sample which comprises the steps of:
(i) contacting the sample with one or more aptamers which binds to the one or more histone modifications;
(ii) detecting or quantifying the binding of said one or more aptamers to the one or more histone modifications in the sample; and
(iii) using the presence or degree of such binding as a measure of the presence of nucleosomes containing the one or more histone modifications in the sample.

According to a further aspect of the invention there is provided a method for detecting the presence of a nucleosome containing one or more histone modifications in a blood, serum or plasma sample which comprises the steps of:
(i) removing, releasing or extracting the one or more histone modifications from the nucleosome complex to produce a free histone modification moiety
(ii) detecting or quantifying each free histone modification in the sample with an aptamer; and
(iii) using the presence or amount of each free histone modification as a measure of the presence of nucleosomes containing the one or more histone modifications in the sample.

According to a further aspect of the invention there is provided a method for detecting the presence of a nucleosome containing one or more histone modifications in a cell which comprises the steps of:
(i) isolating chromatin from a cell;
(ii) digesting, sonicating or otherwise breaking down the chromatin to form mono-nucleosomes and/or oligo-nucleosomes; and
(iii) detecting or measuring the presence of one or more histone modifications in the said nucleosomes according to the method of the first aspect of the invention.

According to a further aspect of the invention there is provided a method for detecting or diagnosing a disease status in an animal or a human subject which comprises the steps of:
(i) detecting or measuring nucleosomes containing one or more histone modifications in a body fluid of a subject according to the method of the first aspect of the invention; and
(ii) using the nucleosome associated histone modification level detected to identify the disease status of the subject.

According to a further aspect of the invention there is provided a method for assessment of an animal or a human subject for suitability for a medical treatment which comprises the steps of:
(i) detecting or measuring nucleosomes containing one or more histone modifications in a body fluid of the subject according to the method of the first aspect of the invention; and
(ii) using the nucleosome associated histone modification level detected as a parameter for selection of a suitable treatment for the subject.

According to a further aspect of the invention there is provided a method for monitoring a treatment of an animal or a human subject which comprises the steps of:
(i) detecting or measuring nucleosomes containing one or more histone modifications in a body fluid of the subject according to the method of the first aspect of the invention;
(ii) repeating step (i) on one or more occasions; and
(iii) using any changes in the nucleosome associated histone modification level detected as a parameter for any changes in the condition of the subject.

According to a further aspect of the invention there is provided a kit for the detection of one or more nucleosome associated histone modifications which comprises one or more aptamers specific for the one or more histone modifications or component part thereof, together with instructions for use of the kit.

### BRIEF DESCRIPTION OF THE FIGURES

FIG. 1: ELISA dose response curves for the detection of human cell free nucleosomes prepared by a published method (*Holdenrieder et al, 2001) containing histone modification H4K16Ac diluted into calf serum. A biotinylated, commercially available antibody is compared with an aptamer biotinylated at the 3′ and 5′ end respectively compared to those present in the serum of a healthy volunteer and a cancer patient.

FIG. 2: Comparison of relative absorption of a commercial H4K16Ac specific monoclonal antibody and 3′-biotinylated H4K16Ac specific aptamer

### DETAILED DESCRIPTION OF THE INVENTION

According to a first aspect of the invention there is provided a method for detecting the presence of a nucleosome containing one or more histone modifications in a sample which comprises the steps of:
(i) contacting the sample with one or more aptamers which binds to the one or more histone modifications;
(ii) detecting or quantifying the binding of said one or more aptamers to the one or more histone modifications in the sample; and
   using the presence or degree of such binding as a measure of the presence of nucleosomes containing the one or more histone modifications in the sample.

The application of immunoassay techniques for the detection of proteins in general depends on the ability to develop affinity reagents with high affinity and specificity for their targets. Antibodies are the most common affinity reagents however their availability for post-translational modifications on proteins is limited due to the close structural similarities between modifications (Williams et al, 2009).

Data is presented herein which provides evidence for the potential of a high degree of sensitivity and specificity using aptamers for detection of single or multiple PTMs within intact nucleosomes. There have been no previous reports of aptamers applied to the detection of cell free nucleosomes containing single or multiple PTMs in blood or any other medium and no such measurements have been suggested or contemplated. In one embodiment, the method comprises the use of a single aptamer to detect a single histone modification. In an alternative embodiment, the method comprises the use of multiple (i.e. more than one) aptamers to detect multiple (i.e. more than one) histone modifications.

The data presented herein reports methods for such tests and their use in plasma and serum samples taken from healthy and diseased subjects. Surprisingly we have shown that high levels of intact nucleosomes comprising specific histone modifications can be detected in plasma and serum samples for which no nucleosomes, or low levels, are detected by nucleosome ELISA methods of the current art.

In one embodiment, the nucleosome is a mononucleosome or oligonucleosome.

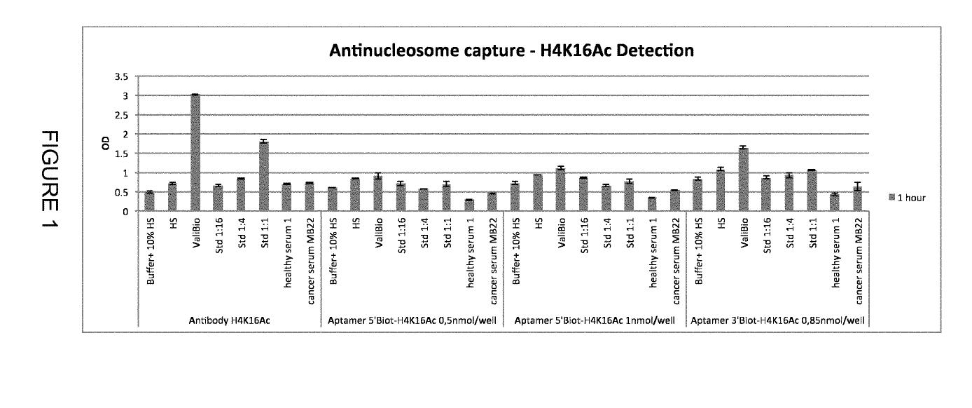

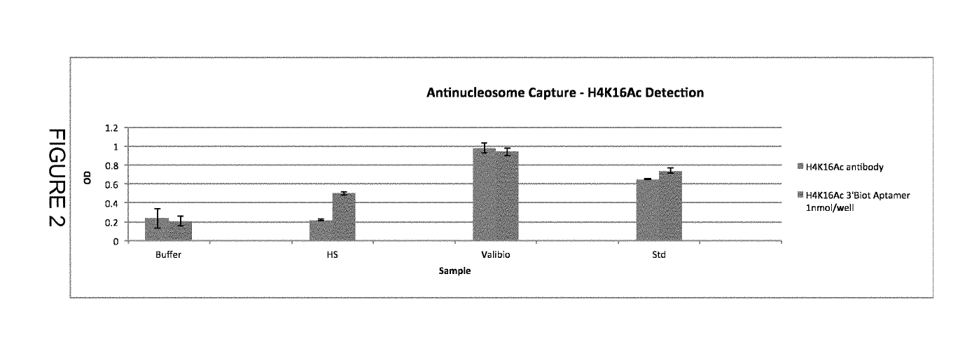

We have developed ELISA tests for the detection and measurement of nucleosomes containing the histone PTM H4K16Ac. An anti-nucleosome antibody was used as capture antibody for these assays in combination with an appropriate specific anti-histone modification aptamer as detection antibody. Results provided herein demonstrated that an aptamer evolved in vitro successfully bound to the H4K16Ac PTM. Such binding was independent of whether the aptamer was biotinylated for detection at the 3′ or 5′ end as shown in FIG. 1. Furthermore, when the higher order structure of the aptamer was removed through a thermal denaturation step, the aptamer was found to perform analogously to a commercially available antibody (Millipore mouse monoclonal Anti-acetyl Histone H4 (Lys16) Antibody, clone 4E10.2; results shown in FIG. 2).

Thus, in one embodiment the aptamer is a denatured aptamer such that the higher order structure has been removed. Such denaturation may typically involve a heat cycle, for example, heating at 95° C. for 1 minute followed by cooling for 10 minutes. In a further embodiment, the aptamer is a DNA aptamer. In an alternative embodiment, the aptamer is an RNA aptamer. In an alternative embodiment, the aptamer is a peptide aptamer.

In one embodiment the post translational modifications detected on a histone protein using the methods of the invention include, but are not limited to, acetylation of a lysine residue, mono-methylation, di-methylation or tri-methylation of a lysine residue; citrullination of a lysine residue; sumolyation of a lysine residue; ubiquitinylation of a lysine residue; ADP-ribosylation of a lysine residue; mono-methylation of an arginine residue, asymmetric or symmetric dimethylation of an arginine residue; methylation of an alanine residue; glycosylation of a serine or threonine residue; phosphorylation of a serine or threonine residue; and isomerisation of a proline residue.

In a further embodiment, the histone modification is selected from H4K16Ac or K3H8Me2sym. In a yet further embodiment, the histone modification is selected from H4K16Ac.

We have shown that ELISA methods work with alternative anti-nucleosome capture antibodies. We have also used the assays to show that nucleosomes containing specific histone modifications can be measured in blood samples taken from diseased subjects.

To investigate levels of nucleosomes found in healthy subjects using the methods of the current art we measured nucleosomes in serum and plasma samples, taken from the healthy subjects. The level of H4K16Ac modified nucleosomes was found to be lower in the serum of a single healthy donor compared to the level in horse serum. The level of H4K16Ac was also shown to be lower than that in combined plasma samples from at least three donors. Combined plasma samples are known to contain elevated nucleosome levels and have been used as a positive control and standard for nucleosome quantification ELISAs (*Holdenrieder et al, 2001).

To investigate levels of nucleosomes found in disease subjects using the methods of the invention we measured nucleosomes containing the H4K16Ac histone modifications in the sera of cancer subjects using either a 3′ or 5′ biotinylated aptamer shown to be specific for an N-terminal peptide from histone H4 containing the H4K16Ac modification. The serum results showed that the level of H4K16 was higher than in the control sample when the 3′-biotinylated aptamer was used for detection compared to both the 5′ modified aptamer and a commercially available antibody (FIG. 1).

We investigated whether removal of higher order structure from the aptamer would improve the performance of the aptamer and found that heat denaturing improved the relative performance of the 3′ aptamer in a serum ELISA compared to a commercially available monocolonal antibody against the H4K16Ac target.

The variability in structure of cell-free nucleosomes in terms of types of histone modifications detected using ELISA methods of the can be normalised as a proportion of total nucleosomes as determined by the level of a non-variable histone such as H4. Alternatively the variability in terms of types of histone modifications detected using ELISA methods of the can be normalised as a proportion of nucleosome associated 5-methylcytosine (5mc) methylated DNA levels and expressed relative to the mean proportions found in healthy subjects. Thus nucleosome structure profiles can be used as a diagnostic tool for the detection, prognosis prediction, monitoring and therapeutic efficacy prediction in cancer and other diseases.

We conclude that the method of the present invention is a successful method for the detection and measurement of nucleosomes containing specific histone modifications. The methods of the invention thus employed have advantages over methods for measuring modified nucleosomes of the current art. It will be clear to those skilled in the art that the methods of the invention can be used to detect and measure nucleosomes directly in any samples where they occur, for example in samples obtained by digestion of chromatin extracted from cells or in biological fluids such as blood, serum or plasma samples. It will also be clear that the methods described here can be developed for any histone modification which an aptamer can be produced.

It will be clear to those skilled in the art that the clinical performance of the invention may be improved further by inclusion of further nucleosome structure tests and by examination of the ratios of different nucleosome structures present.

We have run samples with the method of the invention using a capture antibody specific to intact nucleosomes. A variety of antibodies or other binders may be employed in the invention as a capture binding agent which binds to nucleosomes. These include binding agents directed to bind to epitopes that occur in intact nucleosomes and not in free histones (for example; an epitope found at the junction between two histones in a nucleosome) and also binding agents directed to any nucleosome component including common nucleosome protein, histone or nucleic acid epitopes.

It will be clear to those skilled in the art that the methods of the invention described include a variety of embodiments including classical competitive immunoassays as well as biosensor type assays and label-free assays of the type marketed for example by ForteBio Incorporated of USA which may be immunometric in nature. For example, in one embodiment, the method of the invention may typically involve an immunoassay selected from an immunometric immunoassay (such as a label-free immunometric immunoassay), a competitive immunoassay or a sandwich immunoassay.

According to a further aspect of the invention there is provided a method for detecting the proportion of nucleosomes that comprise one or more histone modifications in a sample comprising the steps of:
(i) detecting or measuring the level of nucleosomes in a sample;
(ii) detecting or measuring the level of one or more nucleosome associated histone modifications according to the method of the first aspect of the invention; and
(iii) using the two measurements to determine the proportion of nucleosomes that contain the one or more histone modifications.

According to one embodiment of this aspect of the invention; both the total nucleosome level in the sample and the nucleosome associated histone modification level of interest are measured using the method of the invention. In another embodiment nucleosome ELISA methods of the current art are used to determine total nucleosome levels. In yet another embodiment a measure of total DNA is used as a proxy for total nucleosome level.

According to another aspect of the invention there is provided a method for detecting or diagnosing the presence of a disease by measuring or detecting the presence and/or the level or concentration of cell free nucleosomes containing one or more histone modifications in a body fluid, and using the detected level as a biomarker of the disease status of a subject including, without limitation, a clinical diagnosis of a disease, a differential diagnosis of disease type or subtype, or a disease prognosis, or a disease relapse, or a diagnosis of subject susceptibility to treatment regimens. It will be appreciated by those skilled in the art that body fluids used for diagnostic testing include without limitation blood, serum, plasma, urine, cerebrospinal fluid and other fluids. In a preferred embodiment the body fluid selected as the sample is blood, serum or plasma. The assay response level, concentration or quantity of a nucleosome associated histone modification in a body fluid may be expressed in absolute terms or relative terms, for example, without limitation, as a proportion of the total nucleosome or total DNA level present or as a ratio to the level of nucleosomes containing another histone PTM or variant or nucleotide.

According to a further aspect of the invention there is provided a method for detecting the presence of a nucleosome containing one or more histone modifications in a cell which comprises the steps of:
(i) isolating chromatin from a cell;
(ii) digesting, sonicating or otherwise breaking down the chromatin to form mono-nucleosomes and/or oligo-nucleosomes; and
(iii) detecting or measuring the presence of one or more histone modifications in the said nucleosomes according to the method of the first aspect of the invention.

It will be appreciated by those skilled in the art that the described method of detecting nucleosome associated histone modifications in cells or tissues is simpler, faster, cheaper, more quantitative and/or more reproducible than currently used methods including IHC, Western Blotting or FACS. The level, concentration or quantity of a particular nucleosome associated histone modification may be expressed in absolute terms or relative terms, for example as a proportion of the total nucleosomes or total DNA present or as a ratio to the level of nucleosomes containing another histone PTM or variant or nucleotide.

It will also be clear that the term nucleosomes is intended to include mononucleosomes and oligonucleosomes and any such chromatin fragments that can be analysed in fluid media.

According to a further aspect of the invention there is provided a kit for the detection of one or more nucleosome associated histone modifications which comprises one or more aptamers specific for the one or more histone modifications or component part thereof, together with instructions for use of the kit.

According to a further aspect of the invention there is provided the use of a kit for the detection of one or more nucleosome associated histone modifications which comprises one or more aptamers specific for the one or more histone modifications or component part thereof, together with instructions for use of the kit.

According to a further aspect of the invention there is provided a method for identifying one or more histone modification biomarkers for assessing the prognosis of a diseased animal or human subject which comprises the steps of:
(i) detecting or measuring the level of cell free nucleosomes containing one or more histone modifications in a body fluid of diseased subjects in accordance with the method of the first aspect of the invention; and
(ii) correlating the level of cell free nucleosomes containing one or more histone modifications detected in a body fluid of diseased subjects with the disease outcome of the subjects.

According to a further aspect of the invention there is provided a method for identifying one or more histone modification biomarkers to be used for the selection of a treatment regimen for a diseased animal or human subject in need of treatment which comprises the steps of:
(i) detecting or measuring the level of cell free nucleosomes containing one or more histone modifications in a body fluid of diseased subjects in accordance with the method of the first aspect of the invention; and
(ii) correlating the level of cell free nucleosomes containing one or more histone modifications detected in a body fluid of diseased subjects with the observed efficacy of a treatment regimen in those subjects.

According to a further aspect of the invention there is provided a method for identifying one or more histone modification biomarkers to be used for monitoring the treatment of a diseased animal or human subject which comprises the steps of:
(i) detecting or measuring the level of cell free nucleosomes containing one or more histone modifications in a body fluid of a diseased subject in accordance with the method of the first aspect of the invention;
(ii) repeating step (i) on one or more occasions during the disease progression of the subject; and
(iii) correlating the level of cell free nucleosomes containing one or more histone modifications detected in a body fluid of a diseased subject with the disease progression in the subject.

According to a further aspect of the invention, there is provided a biomarker identified by the method as defined herein.

It will be clear to those skilled in the art that cell free nucleosomes containing a histone modification can also be detected in a biological fluid including blood, plasma, serum and urine by a procedure involving the extraction of the histone modified protein from the nucleosome complex followed by a method for the detection or quantification of the extracted free histone modified protein. Suitable extraction procedures include commonly used acid extraction procedures for histones which utilise the basic nature of histones proteins. The detection of the free histone modification may be performed, for example, by an immunoassay for the free histone moiety. Thus in one embodiment of the invention a histone protein bearing a post translational modification is extracted from a biological fluid including blood, plasma, serum and urine and the extract is tested for the presence of a histone modification.

Thus in one embodiment of the invention a histone modification is extracted from a biological fluid including blood, plasma, serum and urine and the extract is tested for the presence of a histone modification.

A further aspect of the invention provides ligands or binders, such as naturally occurring or chemically synthesised compounds, capable of specific binding to the biomarker. A ligand or binder according to the invention may comprise a peptide, an antibody fragment, or a synthetic ligand such as a plastic antibody, or an aptamer or oligonucleotide, capable of specific binding to the biomarker. The antibody fragment can be derived from a monoclonal antibody capable of specific binding to the biomarker. A ligand according to the invention may be labeled with a detectable marker, such as a luminescent, fluorescent, enzyme or radioactive marker; alternatively or additionally a ligand according to the invention may be labeled with an affinity tag, e.g. a biotin, avidin, streptavidin or His (e.g. hexa-His) tag. Alternatively ligand binding may be determined using a label-free technology for example that of ForteBio Inc.

A biosensor according to the invention may comprise the biomarker or a structural/shape mimic thereof capable of specific binding to a ligand or binder against the biomarker. Also provided is an array comprising a ligand or mimic as described herein.

Also provided by the invention is the use of one or more ligands as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify the biomarker. In these uses, the detection and/or quantification can be performed on a biological sample as defined herein.

Diagnostic or monitoring kits are provided for performing methods of the invention. Such kits will suitably comprise a ligand according to the invention, for detection and/or quantification of the biomarker, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein.

Biomarkers for detecting the presence of a disease are essential targets for discovery of novel targets and drug molecules that retard or halt progression of the disorder. As the level of the biomarker is indicative of disorder and of drug response, the biomarker is useful for identification of novel therapeutic compounds in in vitro and/or in vivo assays. Biomarkers of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide according to the invention; or the use of a biosensor according to the invention, or an array according to the invention; or a kit according to the invention, to identify a substance capable of promoting and/or of suppressing the generation of the biomarker.

Also there is provided a method of identifying a substance capable of promoting or suppressing the generation of the biomarker in a subject, comprising administering a test substance to a subject animal and detecting and/or quantifying the level of the biomarker present in a test sample from the subject.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

The terms “detecting” and “diagnosing” as used herein encompass identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), and reducing relapse rates.

In one embodiment, said biomarker is released from the cells of a tumour. Thus, according to a further aspect of the invention there is provided a method for the detection of a tumour growth which comprises the steps of (i) measuring a biomarker in a biological sample that is associated with or released from the cells of a tumour and (ii) demonstrating that the level of said biomarker is associated with the size, stage, aggressiveness or dissemination of the tumour.

It is known that increased cell turnover, cell death and apoptosis lead to increased circulatory levels of cell free nucleosomes (Holdenrieder et al, 2001). Circulating cell free nucleosomes level is a non-specific indicator and occurs in a variety of conditions including inflammatory diseases, a large variety of benign and malignant conditions, autoimmune diseases, as well as following trauma or ischaemia (Holdenrieder et al 2001). It will be clear to those skilled in the art that the invention will have application in a variety of disease areas where circulating nucleosomes have been found in subjects. These include, without limitation, trauma (for example; severe injury or surgery), extreme exercise (for example running a marathon), stroke and heart attack and sepsis or other serious infection. We have used the immunoassay method of the invention to measure nucleosome levels and investigate their histone and nucleotide structure variability in a variety of such diseases including cardiomyopathy, systemic lupus erythematosus, ulcerative colitis, chronic obstructive pulmonary disease, Crohn's disease and rheumatoid arthritis and compared these with the results of healthy subjects. We can detect nucleosomes and determine their relative structures (in terms of histone and nucleotide composition) in all these diseases. As methods of the current invention are capable of detection of a wider range of nucleosomes than current nucleosome ELISA methods, the methods of the invention have applications in a wide range of cancer and non-cancer disease areas.

The immunoassays of the invention include immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays and competitive immunoassay methods including labelled antigen and labelled antibody competitive immunoassay methods with a variety of label types including radioactive, enzyme, fluorescent, time-resolved fluorescent and particulate labels. All of said immunoassay methods are well known in the art, see for example Salgame et al, 1997 and van Nieuwenhuijze et al, 2003.

In one embodiment, said biological sample comprises a body fluid. For example, biological samples that may be tested in a method of the invention include cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. Biological samples also include specimens from a live subject, or taken post-mortem. The samples can be prepared, for example where appropriate diluted or concentrated, and stored in the usual manner.

In one embodiment, the method of the invention is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission.

Thus, the invention also provides a method of monitoring efficacy of a therapy for a disease state in a subject, suspected of having such a disease, comprising detecting and/or quantifying the biomarker present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

Thus, according to a further aspect of the invention, there is provided a method for monitoring efficacy of therapy for a disease state in a human or animal subject, comprising:
(i) quantifying the amount of the biomarker as defined herein; and
(ii) comparing the amount of said biomarker in a test sample with the amount present in one or more control(s) and/or one or more previous test sample(s) taken at an earlier time from the same test subject.

A change in the level of the biomarker in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment the monitoring of more rapid changes due to fast acting therapies may be conducted at shorter intervals of hours or days.

According to a further aspect of the invention, there is provided a method for identifying a biomarker for detecting the presence of a disease state. The term “identifying” as used herein means confirming the presence of the biomarker present in the biological sample. Quantifying the amount of the biomarker present in a sample may include determining the concentration of the biomarker present in the sample. Identifying and/or quantifying may be performed directly on the sample, or indirectly on an extract therefrom, or on a dilution thereof.

In alternative aspects of the invention, the presence of the biomarker is assessed by detecting and/or quantifying antibody or fragments thereof capable of specific binding to the biomarker that are generated by the subject's body in response to the biomarker and thus are present in a biological sample from a subject having a disease state.

Identifying and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the biomarker in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand or binder may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing or monitoring according to the invention may comprise analysing a sample by SELDI TOF or MALDI TOF to detect the presence or level of the biomarker. These methods are also suitable for clinical screening, prognosis, monitoring the results of therapy, identifying patients most likely to respond to a particular therapeutic treatment, for drug screening and development, and identification of new targets for drug treatment.

Identifying and/or quantifying the analyte biomarkers may be performed using an immunological method, involving an antibody, or a fragment thereof capable of specific binding to the biomarker. Suitable immunological methods include sandwich immunoassays, such as sandwich ELISA, in which the detection of the analyte biomarkers is performed using two antibodies which recognize different epitopes on a analyte biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

In one embodiment, one or more of the biomarkers may be replaced by a molecule, or a measurable fragment of the molecule, found upstream or downstream of the biomarker in a biological pathway.

The identification of key biomarkers specific to a disease is central to integration of diagnostic procedures and therapeutic regimes. Using predictive biomarkers appropriate diagnostic tools such as biosensors can be developed; accordingly, in methods and uses of the invention, identifying and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

As used herein, the term “biosensor” means anything capable of detecting the presence of the biomarker. Examples of biosensors are described herein. Biosensors according to the invention may comprise a ligand binder or ligands, as described herein, capable of specific binding to the biomarker. Such biosensors are useful in detecting and/or quantifying a biomarker of the invention.

The biomarker(s) of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

Diagnostic kits for the diagnosis and monitoring of the presence of a disease state are described herein. In one embodiment, the kits additionally contain a biosensor capable of identifying and/or quantifying a biomarker. Suitably a kit according to the invention may contain one or more components selected from the group: a ligand binder, or ligands, specific for the biomarker or a structural/shape mimic of the biomarker, one or more controls, one or more reagents and one or more consumables; optionally together with instructions for use of the kit in accordance with any of the methods defined herein.

The identification of biomarkers for a disease state permits integration of diagnostic procedures and therapeutic regimes. Detection of a biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, not achievable using the current measures.

Furthermore, diagnostic biomarker tests are useful to identify family members or patients with mild or asymptomatic disease or who may be at high risk of developing symptomatic disease. This permits initiation of appropriate therapy, or preventive measures, e.g. managing risk factors. These approaches are recognised to improve outcome and may prevent overt onset of the disorder.

Biomarker monitoring methods, biosensors and kits are also vital as patient monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

The invention will now be illustrated with reference to the following non-limiting examples.

### Example 1

A human blood sample containing cell free nucleosomes from healthy subjects prepared according to the method described by Holdenrieder (*Holdenrieder et al, 2001) was tested using an ELISA for the nucleosome associated histone modification H4K16Ac using a solid phase anti-nucleosome capture antibody that binds intact nucleosomes. An aptamer selectively biotinylated at either the 3′ or 5′ end for the post translational histone modification H4K16Ac was compared with a biotinylated, commercially available monoclonal antibody for detection. The human sample was serially diluted in fetal calf serum and was tested in duplicate in the ELISA undiluted and at dilutions of 1:1, 1:4, and 1:16. Neat fetal calf serum was also run in the ELISA as a control sample containing no cell free nucleosomes. A sample from a healthy volunteer and a cancer patient were included for comparison.

The assay method was as follows: a solution of anti-nucleosome antibody in 0.1 M phosphate buffer pH 7.4 was added to microtitre wells (100 μL/well) and incubated overnight at 4° C. to coat the wells with capture antibody. Excess anti-histone antibody was decanted. A solution of bovine serum albumin (20 g/L) was added to the wells (150 μL/well) and incubated 60 minutes at room temperature to block excess protein binding sites on the wells. Excess bovine serum albumin solution was decanted and the wells were washed twice with wash buffer (200 μL/well, 0.05M TRIS/HCl buffer pH 7.5 containing 1% Tween 20). Sample (10 μL/well) and assay buffer (50 μL/well, 0.05M TRIS/HCl pH 7.5 containing 0.9% NaCl, 0.05% sodium deoxycholate and 1% Nonidet P40 substitute) were added to the wells and incubated 90 minutes at room temperature with mild agitation. The sample and assay buffer mixture was decanted and the wells were washed three times with wash buffer (200 μL/well). In the first series of wells a solution of biotinylated monoclonal anti-histone modification H4K16Ac detection antibody was added (50 μL/well). In the second series of wells a solution of 5′-biotinylated H4K16Ac detection aptamer was added (50 μL/well, 0.5 nmol/well). In the third series of wells a solution of 5′-biotinylated H4K16Ac detection aptamer was added (50 μL/well, 1.0 nmol/well). In the fourth series of wells a solution of 3′-biotinylated H4K16Ac detection aptamer was added (50 μL/well, 0.85 nmol/well). The plate was incubated for 90 minutes at room temperature with mild agitation. Excess detection antibody was decanted and the wells were again washed three times with wash buffer (200 μL/well). A solution containing a streptavidin-horse radish peroxidase conjugate was added (50 μL/well) and incubated for 30 minutes at room temperature with mild agitation. Excess conjugate was decanted and the wells were again washed three times with wash buffer (200 μL/well). A coloured substrate solution (100 μL/well, 2,2′-Azinobis [3-ethylbenzothiazoline-6-sulfonic acid]-diammonium salt) was added and incubated 30 minutes at room temperature with mild agitation. A STOP solution (100 μL/well) containing 1% sodium dodecyl sulphate was added and the optical density (OD) of the wells was measured at a wavelength of 405 nm using a standard microtitre plate reader. A reproducible dose response curve of increasing colour with increasing nucleosome associated histone modification H43K16Ac concentration was observed with a low background signal observed in the absence of nucleosome associated histone modification H4K16Ac (horse serum) in the case of the commercial antibody. The positive ELISA signal indicates that the histone modification H4K16Ac detected by the ELISA is incorporated within a nucleosome as the capture antibody used binds to a conformational epitope within intact nucleosomes and does not bind to the H4 histone or specific histone modification alone.

The results shown in FIG. 1 demonstrate that the aptamer successfully bound to the H4K16Ac PTM. Such binding was independent of whether the aptamer was biotinylated for detection at the 3′ or 5′ end. Heat treatment of the aptamer at 95° C. for 1 minute followed by cooling for 10 minutes to denature higher order structure resulted in an increased detection response equivalent to that generated by the commercially available antibody (Millipore mouse monoclonal Anti-acetyl Histone H4 (Lys16) Antibody, clone 4E10.2; results shown in FIG. 2).

### REFERENCES

Allen et al, A simple method for estimating global DNA methylation using bisulfite PCR of repetitive DNA elements. Nucleic Acids Research: 32(3) e38DOI: 10.1093/nar/gnh032
Bawden et al, Detection of histone modification in cell-free nucleosomes. WO 2005/019826, 2005
Boulard et al, Histone variant macroH2A1 deletion in mice causes female-specific steatosis. Epigenetics & Chromatin: 3(8), 1-13, 2010
Cell Biolabs, Inc. Product Manual for “Global DNA Methylation ELISA Kit (5′-methyl-2′-deoxycytidine Quantitation”, 2011
Dai et al, Detection of Post-translational Modifications on Native Intact Nucleosomes by ELISA. http://www.jove.com/details.php?id=2593 doi: 10.3791/2593. J Vis Exp. 50 (2011).
Deligezer et al, Sequence-Specific Histone Methylation Is Detectable on Circulating Nucleosomes in Plasma. Clinical Chemistry: 54(7), 1125-1131, 2008
Epigentek Group Inc, Methylamp™ Global DNA Methylation Quantification Kit, User Guide, Version 2.0802, 2009
Esteller, Cancer epigenomics: DNA methylomes and histone-modification maps Nature Reviews Genetics: 8, 286-298, 2007
Feinberg and Vogelstein, Hypomethylation distinguishes genes of some human cancers from their normal counterparts. Nature: 301, 89-92, 1983
Grutzmann et al, Sensitive Detection of Colorectal Cancer in Peripheral Blood by Septin 9 DNA Methylation Assay. PLoS ONE: 3(11), e3759. doi:10.1371/journal.pone.0003759, 2008
Hervouet et al, Disruption of Dnmt1/PCNA/UHRF1 Interactions Promotes Tumorigenesis from Human and Mice Glial Cells PLoS ONE: 5(6). e11333. doi:10.1371/journal.pone.0011333, 2010
Herranz and Esteller, DNA methylation and histone modifications in patients with cancer: potential prognostic and therapeutic targets. Methods Mol Biol: 361, 25-62, 2007
*Holdenrieder et al, Nucleosomes in serum of patients with benign and malignant diseases. Int. J. Cancer (Pred. Oncol.): 95, 114-120, 2001
Holdenrieder et al, Nucleosomes in Serum as a Marker for Cell Death. Clin Chem Lab Med: 39(7), 596-605, 2001
Holdenrieder et al, Cell-Free DNA in Serum and Plasma: Comparison of ELISA and Quantitative PCR. Clinical Chemistry: 51(8), 1544-1546, 2005
Holdenreider and Stieber, Clinical use of circulating nucleosomes. Critical Reviews in Clinical Laboratory Sciences: 46(1)′ 1-24, 2009
Hsiung et al, Global DNA Methylation Level in Whole Blood as a Biomarker in Head and Neck Squamous Cell Carcinoma. Cancer Epidemiology, Biomarkers & Prevention: 16(1), 108-114, 2007
Hua et al, Genomic analysis of estrogen cascade reveals histone variant H2A.Z associated with breast cancer progression. Molecular Systems Biology 4: Article number 188; doi:10.1038/msb.2008.25, 2008
Hyun et al, An RNA Aptamer that Selectively Recognizes Symmetric Dimethylation of Arginine 8 in the Histone H3 N-Terminal Peptide. Nucleic Acid Therapeutics: 21, 157-163, 2011
Kapoor et al, The histone variant macroH2A suppresses melanoma progression through regulation of CDK8. Nature: 468, 1105-1111, 2010
Lin et al, In Vitro Selection of Histone H4 Aptamers for Recognition Imaging Microscopy. J. Am. Chem. Soc: 129(47), 14568-14569, 2007
Lin et al, Recognition Imaging of Acetylated Chromatin Using a DNA Aptamer. Biophysical Journal, 97: 1804-1807, 2009
Mansour et al, The Prognostic Significance of Whole Blood Global and Specific DNA Methylation Levels in Gastric Adenocarcinoma. PLoS ONE: 5(12), e15585. doi:10.1371/journal.pone.0015585, 2010
Moore et al, Genomic DNA hypomethylation as a biomarker for bladder cancer susceptibility in the Spanish Bladder Cancer Study: a case-control study. The Lancet Oncology: 9(4), 359-366, 2008
Ogoshi et al, Genome-wide profiling of DNA methylation in human cancer cells. Genomics: 98(4), 280-287, 2011
Pennings et al, DNA methylation, nucleosome formation and positioning. Briefings in functional genomics and proteomics: 3(4), 351-361, 2005
Rodriguez-Paredes and Esteller, Cancer epigenetics reaches mainstream oncology. Nature Medicine: 17(3), 330-339, 2011
Ramos et al, A DNA Aptamer Population specifically Detects Leishmania Infantum H2A Antigen. Laboratory Investigation: 409-416, 2007
Salgame et al, An ELISA for detection of apoptosis. Nucleic Acids Research: 25(3), 680-681, 1997
Sporn et al, Histone macroH2A isoforms predict the risk of lung cancer recurrence. Oncogene: 28(38), 3423-8, 2009
Stroud et al, 5-Hydroxymethylcytosine is associated with enhancers and gene bodies in human embryonic stem cells. Genome Biology: 12:R54, 2011
Tachiwana et al, Structures of human nucleosomes containing major histone H3 variants. Acta Cryst: D67, 578-583, 2011
van Nieuwenhuijze et al, Time between onset of apoptosis and release of nucleosomes from apoptotic cells: putative implications for systemic lupus erythematosus. Ann Rheum Dis: 62: 10-14, 2003
Vasser et al, Measurement of Global DNA Methylation. Genetic Engineering and Biotechnology News: 29(7), 2009
Whittle et al, The Genomic Distribution and Function of Histone Variant HTZ-1 during C. elegans Embryogenesis. PLoS Genet: 4(9), 1-17, 2008
Williams et al, Evolution of a Histone H4-K16 Acetyl-Specific DNA Aptamer. J. Am. Chem. Soc: 131, 6330-6331, 2009
Yu et al, Aptamers can Discriminate Alkaline Proteins with High Specificity. Chembiochem: 12(17), 2659-2666, 2011
Zee et al, Global turnover of histone post-translational modifications and variants in human cells. Epigenetics & Chromatin: 3(22), 1-11, 2010
Zhang et al, Analysis of global DNA methylation by hydrophilic interaction ultra high-pressure liquid chromatography tandem mass spectrometry. Analytical Biochemistry: 413(2), 164-170, 2011

1. A method for detecting the presence of a nucleosome containing one or more histone modifications in a sample which comprises the steps of:
(i) contacting the sample with one or more denatured aptamers which binds to the one or more histone modifications;
(ii) detecting or quantifying the binding of said one or more denatured aptamers to the one or more histone modifications in the sample; and
(iii) using the presence or degree of such binding as a measure of the presence of nucleosomes containing the one or more histone modifications in the sample.

2. The method of claim 1, wherein the aptamer is a DNA aptamer an RNA aptamer or a peptide aptamer.

3. The method of claim 1, wherein the nucleosome is a mononucleosome or an oligonucleosome.

4. The method of claim 1, wherein the sample is a biological fluid, such as blood serum or plasma.

5. The method of claim 1, wherein the one or more histone modification is selected from: acetylation of a Lysine residue; mono-methylation, di-methylation or tri-methylation of a lysine residue; mono-methylation, symmetric or asymmetric di-methylation of an arginine residue, phosphorylation of a threonine, serine residue; citrullination of an arginine residue, ubiquitinylation, sumoylation; and glycosylation of a serine or threonine residue.

6. The method of claim 5, wherein the histone modification is selected from H4K16Ac or H3R8Me2sym.

7. A method for detecting the presence of a nucleosome containing one or more histone modifications in a blood, serum or plasma sample which comprises the steps of:
(i) removing, releasing or extracting the one or more histone modifications from the nucleosome complex to produce a free histone modification moiety;
(ii) detecting or quantifying each free histone modification in the sample with a denatured aptamer; and
(iii) using the presence or amount of each free histone modification as a measure of the presence of nucleosomes containing the one or more histone modifications in the sample.

8. A method for detecting the presence of a nucleosome containing one or more histone modifications in a cell which comprises the steps of:
(i) isolating chromatin from a cell;
(ii) digesting, sonicating or otherwise breaking down the chromatin to form mono-nucleosomes and/or oligo-nucleosomes; and
(iii) detecting or measuring the presence of one or more histone modifications in the said nucleosomes according to the method as defined in claim 1.

9. A method for detecting or diagnosing a disease status in an animal or a human subject which comprises the steps of:
(i) detecting or measuring nucleosomes containing one or more histone modifications in a body fluid of a subject according to the method as defined in claim 1; and
(ii) using the nucleosome associated histone modification level detected to identify the disease status of the subject.

10. The method of claim 9, wherein the disease is cancer and said cancer is a cancer of the bladder, breast, colon, cervix, esophagus, kidney, large intestine, lung, oral cavity, ovary, pancreas, prostate, rectum, skin or stomach.

11. The method of claim 10, wherein the cancer is a cancer of the colon, lung, oral cavity or pancreas.

12. A method for assessment of an animal or a human subject for suitability for a medical treatment which comprises the steps of:
(i) detecting or measuring nucleosomes containing one or more histone modifications in a body fluid of the subject according to the method as defined in claim 1; and
(ii) using the nucleosome associated histone modification level detected as a parameter for selection of a suitable treatment for the subject.

13. A method for monitoring a treatment of an animal or a human subject which comprises the steps of:
(i) detecting or measuring nucleosomes containing one or more histone modifications in a body fluid of the subject according to the method as defined in claim 1;
(ii) repeating step (i) on one or more occasions; and
(iii) using any changes in the nucleosome associated histone modification level detected as a parameter for any changes in the condition of the subject.

14. A kit for the detection of one or more nucleosome associated histone modifications wherein said kit comprises one or more denatured aptamers specific for the one or more histone modifications or component part thereof, together with instructions for use of the kit.

15. A method for identifying one or more histone modification biomarkers for assessing the prognosis of a diseased animal or human subject which comprises the steps of:
(i) detecting or measuring the level of cell free nucleosomes containing one or more histone modifications in a body fluid of diseased subjects in accordance with the method as defined in claim 1; and
(ii) correlating the level of cell free nucleosomes containing one or more histone modifications detected in a body fluid of diseased subjects with the disease outcome of the subjects.
